(19) United States
(12) Patent Application Publication
(10) Pub. No.: US 2025/0191690 A1
(43) Pub. Date: Jun. 12, 2025

(54) METHODS FOR THE ALIGNMENT OF SEQUENCE READS TO NONACYCLIC GENOME GRAPHS ON HETEROGENEOUS COMPUTING SYSTEM

(71) Applicant: POLITECNICO DI MILANO, Milano (IT)

(72) Inventors: Guido Walter DI DONATO, Milano (IT); Alberto ZENI, Milano (IT); Mirko COGGI, Milano (IT); Guglielmo BRUNO, Milano (IT); Marco Domenico SANTAMBROGIO, Milano (IT)

(21) Appl. No.: 18/845,353

(22) PCT Filed: Mar. 7, 2023
(86) PCT No.: PCT/EP2023/055791

§ 371 Date: Sep. 9, 2024

(30) Foreign Application Priority Data
Mar. 9, 2022 (IT) 102022000004439

(51) Int. Cl. G16B 30/10; G16B 45/00; G16B 50/30

(52) U.S. Cl. CPC G16B 30/10; G16B 45/00; G16B 50/30

(57) ABSTRACT

The disclosed embodiments concern methods, apparatus, systems, and computer program products for computing the best alignment score between a set of query sequences and a non-acyclic sequence-labelled reference genome graph comprising a processor and non-transitory memory. In an embodiment, said computing is performed employing an alignment algorithm suitable for non-acyclic graph that leverages inter-sequence and intra-sequence parallelism, executed on a heterogeneous computing system, containing at least a CPU (Central Processing Unit) and a GPU (Graphics Processing Units).

The present invention concerns methods, apparatus, systems, and computer program products for computing the best alignment score between a set of query sequences and a non-acyclic sequence-labelled reference genome graph.

### STATE OF THE ART

Next Generation Sequencing (NGS) technologies produced an explosion in the amount of available genomic data, which has been extremely valuable to the development of genomic research and personalized medicine. Moreover, NGS has enabled studies that would have been impossible before. Examples are population genomics and pangenomics, promising branches of computational genomics focusing on the analysis of population genomes and its generalization, the pangenome, that is the entire sets of genes and other regulatory sequences for all strains within a species or clade, including the central genome and all its possible variants.

The emergence of these new studies highlighted how scaling up established bioinformatics pipelines would not be sufficient to fully leverage the inter-individual and intra-individual variability of genomic data. Novel, more efficient computational paradigms are needed.

A promising solution has been identified in graph-based data structures.

A “genome graph” is defined as a directed graph made of a set of nodes containing genomic sequences, and a set of edges connecting couples of nodes. Each directed edge connects a source node to a target node, with a specific overlap between the last characters of the source node's sequence, and the first characters of the target node's sequence. A “null overlap” means that the first character of the target node directly follows the last character of the source node. Thus, in a genome graph, each path, that is an ordered sequence of nodes and edges, encodes a significant sequence in the represented genome or set of genomes.

Several aligners for genome graphs have been proposed to search query sequences in such graphs. These aligners compute the alignment score for each query sequence to evaluate how similar it is to the paths in the graph, but how to efficiently align DNA sequence reads to genome graphs remains an open question.

The properties of the underlying graph representation affect the alignment's accuracy and speed: greater granularity (i.e., less characters per node) and the presence of cycles provide more accurate results, at the cost of longer alignment times.

Non-acyclic graphs allow to represent the inter-individual and intra-individual variability of genomic data in the most accurate way, enabling more accurate analysis. However, processing non-acyclic graphs is much more difficult and compute-intensive than processing acyclic graphs, as they require more complex algorithm capable of handling cyclicity. Existing aligners offer different trade-offs, suitable for different applications, and they rely on two different classes of alignment algorithms.

The first class of currently available aligners leverage the sequence-to-sequence alignment between the query sequences and the nodes' sequences in the genome graph.

The GraphGenome Pipeline, proposed in [1], is a significant example of such an approach. However, this implementation only supports coarse-grained Directed Acyclic Graphs (DAGs), which considerably limits the accuracy of the alignment and the capability of handling complex structural variations.

The Variation Graph (VG) toolkit [2] offers a set of computational methods for creating, manipulating, and using graphs as references. The tool leverages an indexing strategy to perform seed-and-extend alignments, thus it is very efficient but at the expense of an excessive memory footprint. Moreover, it performs a DAG-ification step (i.e., internally represent the genome graph as a DAG) that can significantly degrade the quality of the alignment and the execution time for highly branched non-acyclic graphs.

The second class of algorithms is based on “pure” sequence-to-graph alignment between the characters in the query sequences and those in the genome graph.

PaSGAL [4] proposes the first parallel algorithm for sequence-to-graph alignment that leverages multiple cores and Single Instruction Multiple Data (SIMD) operations to exploit inter-sequence parallelism, that is the parallel execution of the alignment algorithm for multiple sequences. PaSGAL is very efficient both in terms of runtime performance and memory footprint, but the underlying algorithm only works with DAGs and does not support affine gap penalty functions.

GraphAligner [3] supports fine-grained non-acyclic graphs and exact alignment, guaranteeing the best alignment precision. It also leverages intra-sequence parallelism, that is the parallel execution of the operations needed to align one single sequence to the genome graph. However, its exact alignment algorithm has significant memory cost and very long execution time. Moreover, GraphAligner does not support custom scoring matrices and affine gap penalty functions, which limits its flexibility, and its underlying algorithm is not well suited for GPU processing.

AStarix [5, 6] is the result of a recent effort to provide an efficient optimal algorithm for aligning sequences to non-acyclic genome graphs. It is a generalization of Dijkstra algorithm with domain-specific heuristics that result in an optimal alignment algorithm. However, AStarix has a high memory cost, and its execution time scales badly with the number of edits between the query and the reference genome graph. This hinders its applicability to most of real-world applications.

Finally, HGA [7] addresses the acceleration of sequence-to-graph alignment on the GPU. However, the underlying algorithm only works with DAGs, it does not support affine gap penalty functions, and it does not compute the traceback associated to the alignments, that is the sequence of edits the query must undergo to perfectly match the path identified in the graph. Moreover, the approach proposed in HGA only leverages inter-sequence parallelism between different alignment task, but it does not use intra-sequence parallelism to optimize the execution of the single alignment.

Jain et al. [8] introduced a novel algorithm based on the shortest-path search problem, which has better time complexity than state-of-the-art alignment algorithms suitable for non-acyclic graphs. However, the algorithm proposed by Jain et al. [8] is sequential, it does not expose intra-sequence parallelism, and it is not suitable for GPU-based processing.

US20170199960A1, U.S. Pat. No. 10,192,026B2, US20210280272A1, U.S. Pat. Nos. 11,049,587B2, 11,062,793B2 describe sequence-to-graph alignment. However, the described methods are suitable only for DAGs, without supporting non-acyclic genome graphs, which intrinsically limits their accuracy and efficiency when handling complex informative variations like Variable Number Tandem Repeats (VNTRs), Copy Number Variations (CNVs), and other Structural Variations (SVs).

US2020286586A1 describes a method for determining variation in Short Tandem Repeat (STR) regions, based on the alignment of sequence reads to genome graphs with self-loops representing STRs. The method is not suitable for genome graphs encoding whole chromosomes or entire genomes, nor for graphs containing cyclic paths with a depth greater than 1, i.e., not self-loops. Moreover, the underlying alignment strategy still does not handle cycles directly but requires a DAG-ification step that can significantly degrade the quality of the alignment and the execution time for real-world non-acyclic graphs. Furthermore, all the aforementioned methods, except HGA [7], do not provide optimization strategies for multi-threaded heterogeneous computing systems equipped with GPU(s).

Faster and more accurate sequence-to-graph alignment methods are needed to fully realize the potential of graph-based reference representations.

### DESCRIPTION

The invention describes a method for the alignment of sequence reads to non-acyclic sequence-labelled genome graphs, in a preferred embodiment employing heterogeneous computing resources (CPUs and GPUs).

### DRAWINGS

FIG. 1: Overview of an embodiment of the method according to the present invention.

FIG. 2: Graphical Representation of step A of the method according to the invention.

FIG. 3: Example of alignment graph (comparative purposes).

FIG. 4: Example of alignment graph using affine penalty function (comparative purposes).

FIG. 5: Examples of dual graph representations.

FIG. 6: Overview of the heterogeneous computing system for computing sequence-to-graph alignment. The CPU handles the communication with the GPU(s) scheduling threads accordingly to parallelize and equally divide the load on the available resources. Multiple alignments are assigned to multiple GPU(s) Streams to leverage inter-sequence parallelism, each Stream then parallelizes the alignments scheduling multiple GPU Threads to leverage intra-sequence parallelism.

FIG. 7: Overview of a further embodiment of the method according to the invention.

FIG. 8: Overview of a further embodiment of the method according to the invention.

FIG. 9: Overview of a further embodiment of the method according to the invention.

FIG. 10: Sequential algorithm for sequence to graph alignment, comparative purposes.

FIG. 11: Parallel algorithm for sequence to graph alignment according to the invention.

FIG. 12: Examples of sequence to graph alignment leveraging an adaptive bandwidth heuristic to speed up the computation by avoiding exploring inconvenient paths in the alignment graph.

FIG. 13: (A) alignment time scaling w.r.t. the number of nodes; (B) memory usage scaling w.r.t. the number of nodes.

FIG. 14: Alignment time scaling w.r.t the number of employed GPUs.

FIG. 15: (A) alignment time scaling w.r.t. the bandwidth value k of the heuristic; (B) memory usage scaling w.r.t. the bandwidth value k of the heuristic.

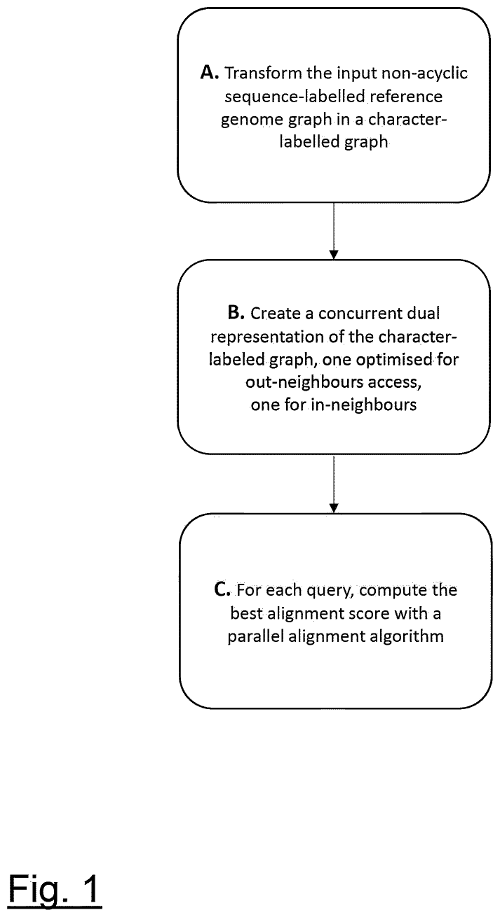

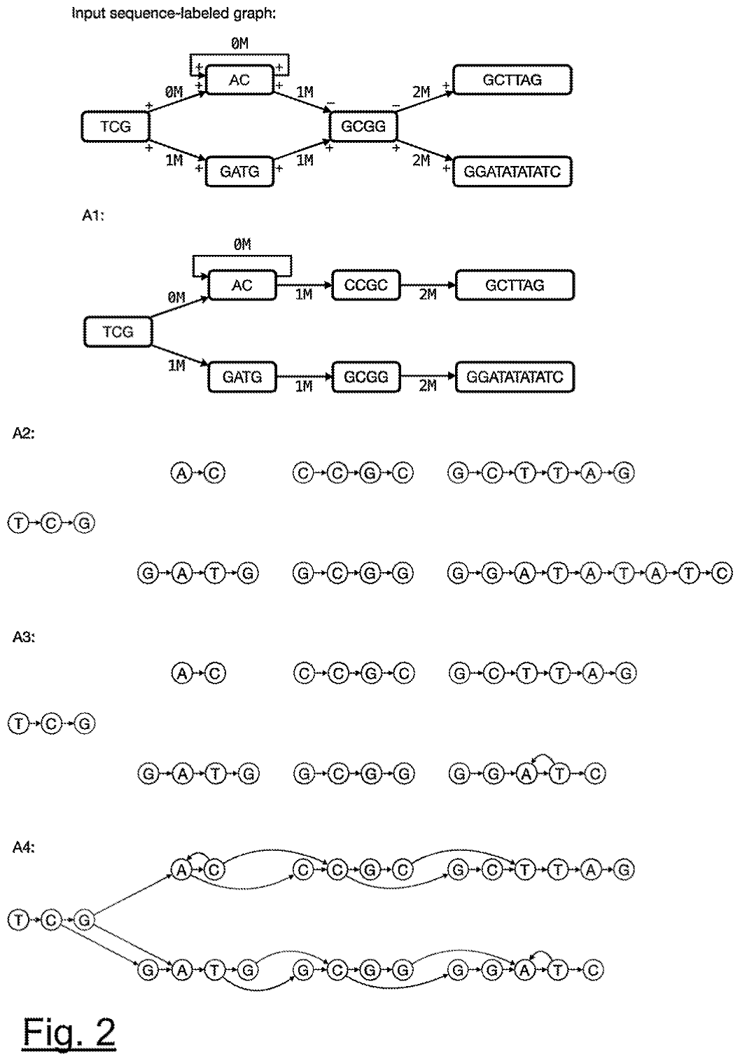

It forms a first object of the present invention, with reference to FIG. 1, a system for computing the best alignment score between a set of query sequences and a non-acyclic sequence-labelled reference genome graph comprising a processor and non-transitory memory, wherein the memory comprises instructions that, when executed, cause the processor to:
A. Transforming the non-acyclic sequence-labelled reference genome graph, which comprises nodes and different paths, in a character-labelled graph. This transformation step enables the best representation of genomic variability, and it comprises the following steps, illustrated in FIG. 2:
   A1: duplicating string-labelled nodes, if any, that appear in different paths, both in their original and reverse complement strands;
To note, step A1, i.e., duplicating string-labelled nodes, accounts for potential occurrences of chromosomal inversion or other complex variations. This is not equivalent to the duplication of each query for both complementary strands before the execution of the alignment. It is often impossible to know which of the two DNA complementary strands a certain query was sequenced from. Thus, a query sequence is usually duplicated by computing its reverse complement sequence and, for completeness, both the sequences are mapped onto the genome graph, which usually encodes only one of the two complementary strands. Surprisingly, Step A1, by duplicating string-labelled nodes, enables the most accurate representation of complex variations that involve inversions of some parts of the DNA sequence.
   A2: unrolling each string-labelled node to form a linear subgraph of contiguous character-labelled nodes;
   A3: analysing the node sequences to search for repeats of subsequences, if any, that can be represented as cycles in the character-labelled sub-graph;
To note, in the input sequence-labelled graph, some nodes could contain sequences that present repetitive motives, e.g., in “ATCGCGCGCGCGTT”, the “CG” subsequence has 5 occurrences. This processing step eventually introduce cycles in some of the character-labelled subgraphs, in order to guarantee the finest granularity, resulting in improved accuracy when handling genetic variations involving repetitions, e.g., tandem repeats, CNVs.
   A4: connecting said character-labelled sub-graphs each other using the information contained in the edges of the non-acyclic sequence-labelled genome graph (source node, target node, overlap) wherein an edge is created between the last character-labelled node extracted from the source node, and the character-labelled node corresponding to the character in the i-th position of the target node's sequence, where i is equal to the length of the overlap+1, then another edge is created between the last character-labelled node preceding the overlap in the source node, and the first character-labelled node of the target node's sequence, obtaining a character-labelled reference graph;
B. Creating a concurrent dual representation of the character-labelled reference graph, made by a single copy of nodes' information, and two copies of edges' information employing two complementary representations that provide concurrent fast access to the in-neighbours and out-neighbours of a certain node, respectively, to enable optimizations that exploit intra-sequence parallelism;
To note, computing the best alignment score requires to navigate the character-labelled reference graph by accessing the neighbours of a certain node repeatedly, which is a time-consuming operation. Each data structure suitable for storing a graph can be populated in two complementary ways: one allows to efficiently access the information about the out-neighbours of each node, i.e., the neighbours connected to the node through outgoing edges, while the other allows to efficiently access the information about the in-neighbours of each node, i.e., the neighbours connected to the node through incoming edges. Thus, employing a concurrent dual representation enables a fast access to both in-neighbours and out-neighbours, which can be leveraged to push the performance of the alignment execution.
C. Computing the alignment score between each query sequence and the dually represented character-labelled reference graph.

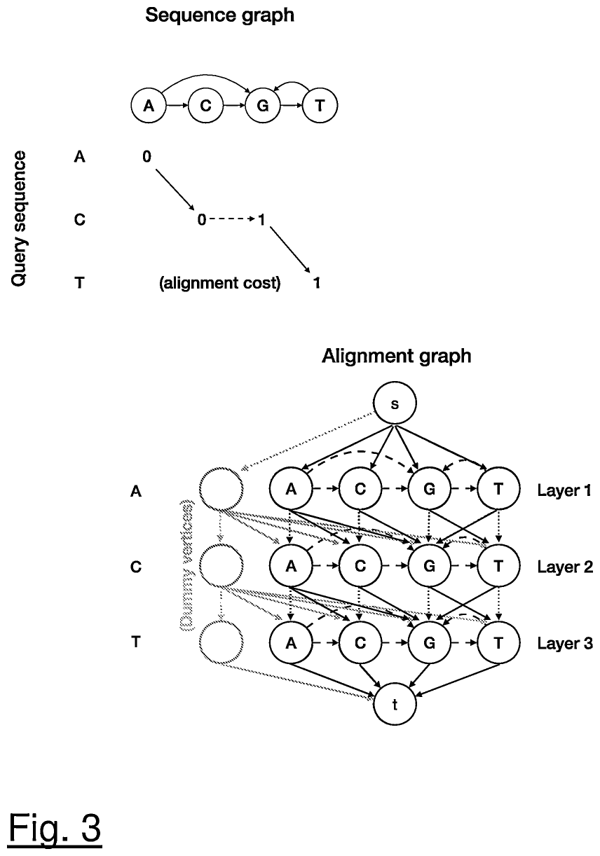

In a preferred embodiment, said computing step C comprises the creation of the alignment graph, composed by a source vertex s, a sink vertex t, and multiple layers given by m repetitions of a character-labelled graph, called sequence graph, where m is the number of characters in the query sequence, as shown in FIG. 3. A column of dummy vertices is added to support semi-global alignment, accommodating the possibility of deleting a prefix of the query sequence. The alignment is performed by searching for the shortest path from the source vertex s to the sink vertex t, leveraging both inter-sequence and intra-sequence parallelism,

In the alignment graph, each weighted edge ω(u, v) connects each node u to its neighbour v, both within a layer or between two consecutive layers. The alignment costs, i.e., edit distance, between the query sequence and the sequence graph, is computed by navigating the alignment graph and assigning to each node a score representing its minimal distance from the source vertex. The links inside a layer (dashed black links in FIG. 3) assign the insertion score (Δᵢₙₛ), while those between two consecutive layers (solid black links) are used for both matches (ω(u, v)=0) and mismatches (Δ_{sub}), depending on the comparison between the character label of each node inside the second layer, and the corresponding character of the query sequence. Moreover, each node is also connected to its copy in the next layer to allow deletions (dotted black links, Δ_{del}). Finally, each dummy vertex is connected to all nodes in the subsequent layer, to enable semi-global alignment.

Finding the shortest path in such an alignment graph allows to compute the alignment score between a query and the character-labelled sequence graph, employing a linear gap penalty function, which assign a penalty to insertions and deletions equal to the gap length L times the associated edit score (i.e., L*Δᵢₙₛ or L*Δ_{del}). In the case of affine gap penalty function, there is an additional penalty for gap opening (Δᵢₙₛ₀, Δ_{del0}), thus the penalty for a certain gap is given by Δᵢₙₛ₀+L*Δᵢₙₛ or Δ_{del0}+L*Δ_{del}, where Δᵢₙₛ and Δ_{del} are called insertion/deletion extension penalties. To compute the sequence-to-graph alignment under the affine penalty function formulation, it is necessary to build the alignment graph differently.

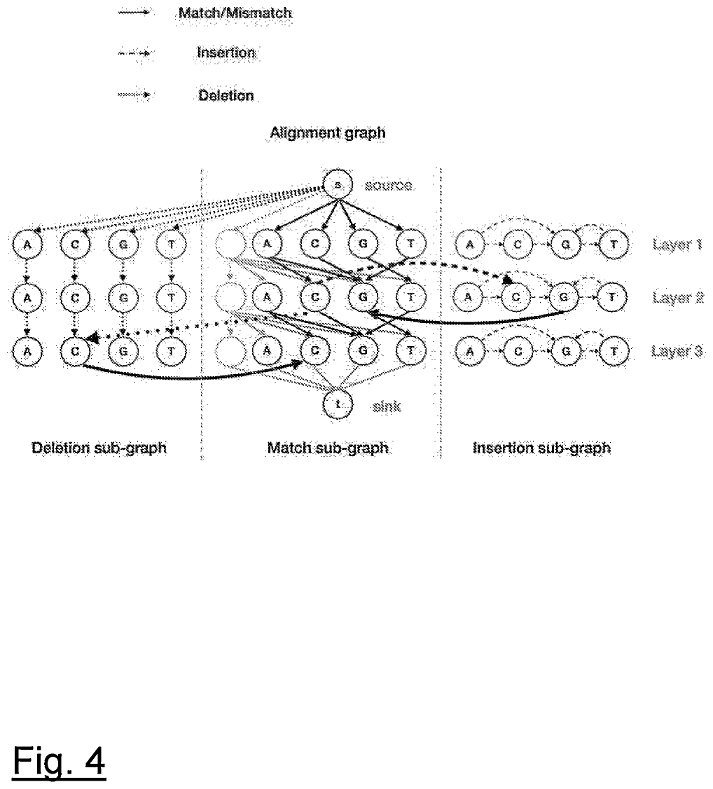

As shown in FIG. 4, in this case the alignment graph contains three sub-graphs with substitution (solid links), deletion extension (dotted links), and insertion extension (dashed links) cost-weighted edges respectively. More edges are added to connect the match sub-graph to the other subgraphs, and their weights are adjusted in such a way that a cost for opening a gap is penalised whenever a path leaves the match sub-graph (dashed and dotted bold links) to either the insertion (Δᵢₙₛ₀) or the deletion sub-graph (Δ_{del0}), while no penalty (i.e., 0) is associated to edges leaving the insertion or deletion sub-graphs to the match sub-graph (solid bold links). This is suitable both in the case of linear and affine penalty function.

The alignment is formulated as a shortest-path problem in the appropriately constructed edge-weighted alignment graph. In fact, formulating the sequence to graph alignment problem as a dynamic programming recursion, while easy for DAGs using topological ordering, is difficult for general graphs due to the possibility of cycles. Instead, formulation as a shortest-path problem in an alignment graph is still rather convenient, even for graphs with cycles. Advantageously, leveraging inter-sequence parallelism, and enabling intra-sequence parallelism reduces the computation time when executed on heterogeneous computing systems.

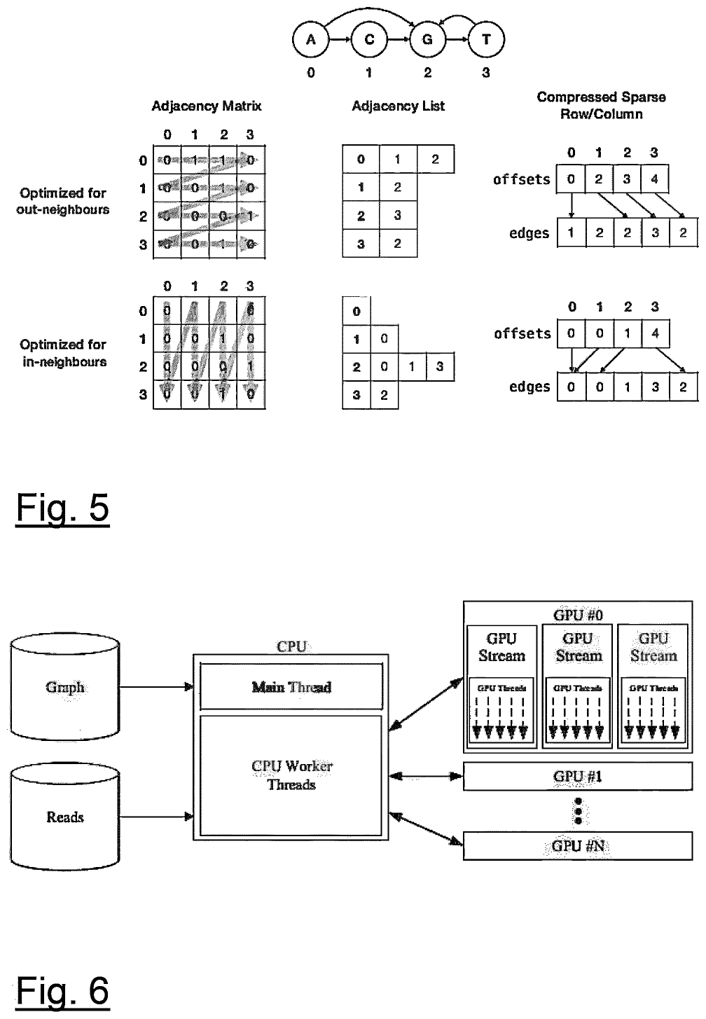

In an embodiment, with reference to FIG. 5, said representations employed in said step B are selected among the group comprising:
an adjacency matrix saved both in a row-major (out-neighbours) and column-major (in-neighbours) order;
two adjacency lists storing, for each node, the list of in-neighbours and the list of out-neighbours;
a compressed adjacency matrix, stored using both the Compressed Sparse Row (CSR) representation (out-neighbours) and the Compressed Sparse Column (CSC) representation (in-neighbours).

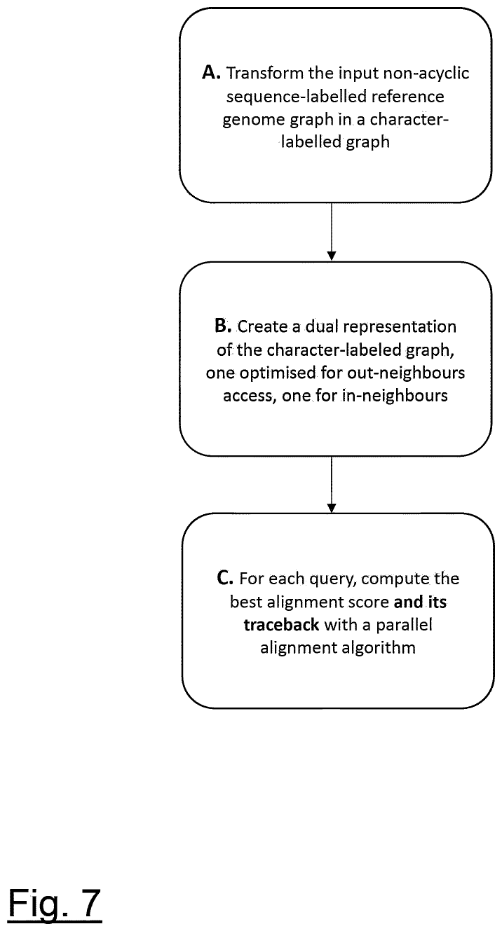

In an embodiment, shown in FIG. 6, said computing step C is performed employing a heterogeneous computing system, containing at least a CPU (Central Processing Unit) and a GPU (Graphics Processing Units). In this embodiment, both the CPU(s) and GPU(s) are used for executing the sequence-to-graph alignment algorithm, employing both inter-sequence parallelism (i.e., parallelism between the alignments of different query sequences) and intra-sequence parallelism (i.e., parallelism between the processes involved in the alignment of each query sequence).

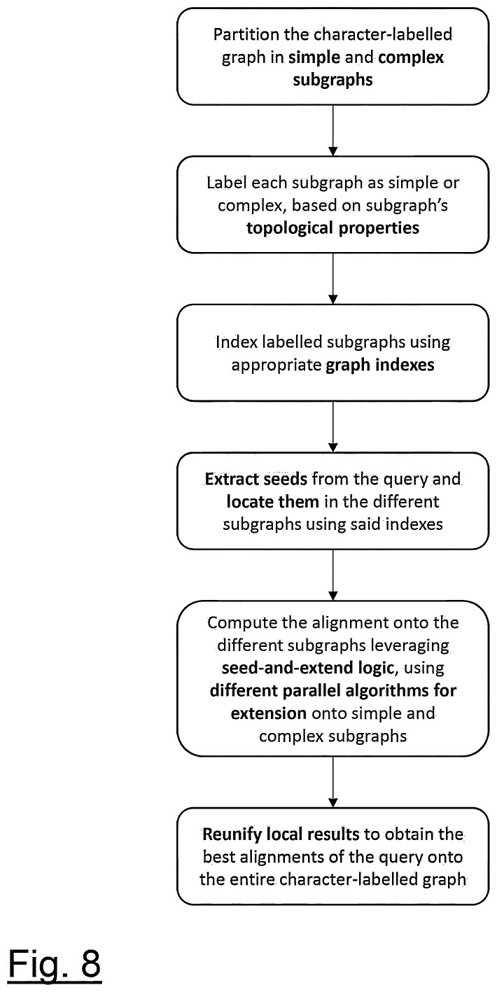

In a further embodiment, according to the flow chart in FIG. 7, said computing step C is employed for computing the best alignments between a set of query sequences and the previously computed non-acyclic character-labelled reference genome graph, and the traceback associated to them. The method gives as output all the information about each best alignment: the alignment score, the start/end positions and the complete path in the graph, i.e., the ordered list of visited nodes for the considered alignment, and a CIGAR string describing the complete sequence of edits the query should undergo to match the sequence extracted by navigating the graph from the start to the end position, through the identified path.

In this embodiment, for each character-labelled node of the alignment graph, the method requires to store, in addition to the edit distance between the query and the shortest path in the alignment graph reaching that node, two fields:
an array named Path, containing the ordered sequence of IDs of the nodes visited in the shortest path culminating to the considered nodes;
a CIGAR string, describing the complete sequence of edits the sub-query, i.e., the subsequence of the query sequence up to position i, where i is the current iteration of the alignment algorithm, should undergo to match the sequence extracted by navigating the graph through the identified Path.

These fields are updated every time the distance of a certain node is updated during the execution of the alignment algorithm based on shortest-path search. The Path of a node is updated by appending the considered node ID to the Path of its predecessor in the shortest path, i.e., the in-neighbour whose distance is being propagated to the current node.

The CIGAR string of a node is updated similarly, by adding to the CIGAR of its predecessor in the shortest path the information about the kind of edge, i.e., the type of edit, connecting the predecessor to the considered node in the alignment graph. If such an edge represents the same kind of edit of the last edit included in the CIGAR, the last counter in the CIGAR must be increased by 1, otherwise a new counter (with value=1) is started, associated to the considered kind of edit.

At the end of the execution, the nodes with the shortest distance, i.e., the best alignment score, will also contain the Path and CIGAR fields, constituting the traceback of the corresponding best alignment.

In this embodiment, the method can be employed in all the applications that require an accurate mapping of certain sequences in a non-acyclic genome graph, providing the complete information about the differences between the query sequences and the paths in the graph they are mapped to.

In an embodiment, said computing step C is executed through a seed-and-extend approach, which consists of four steps:
a. the dually represented character-labelled reference graph is indexed by leveraging graph indexes suitable for non-acyclic graphs, such as the Generalized Compressed Suffix Array index;
b. subsequences called seeds are extracted from each query sequence using a moving window of fixed or variable length onto the query sequence;
c. for each query, candidate alignment locations are retrieved by mapping the previously extracted seeds onto the graph by leveraging previously built graph indexes;
d. the complete alignment of the query sequence is computed by extending the match of all the seeds, or a subset of them, using a parallel alignment algorithm based on shortest-path search in the alignment graph, where the first layer of the alignment graph contains only the nodes corresponding to the last character of each mapped seed, i.e., where only the scores of the alignment graph's nodes corresponding to the last character of the mapped seeds are propagated in the first iteration of the alignment algorithm.

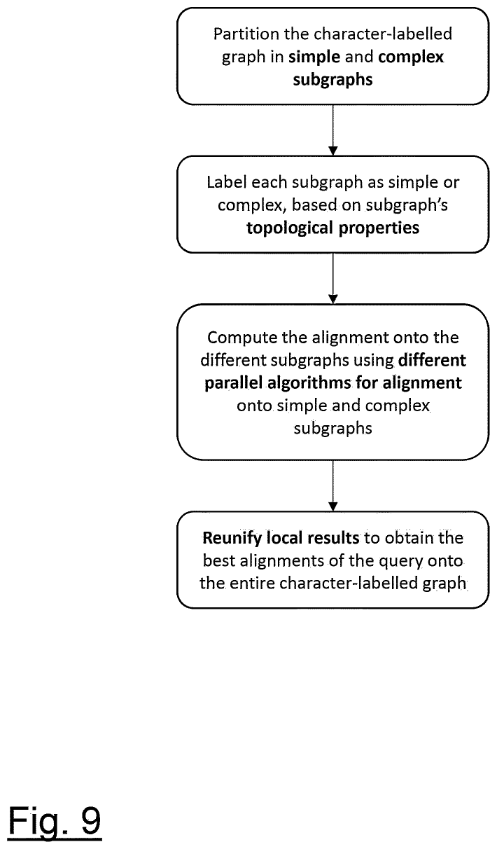

In an embodiment, according to the flow chart in FIG. 8 said computing step C is executed using a local adaptive approach where, before executing the seed-and-extend alignment, the character-labeled graph is segmented (i.e., partitioned) into simple regions and complex regions, i.e., a series of connected subgraphs, where the formers are Directed Acyclic Graphs, and the latter are Directed Non-Acyclic Graphs. After “labeling” the different subgraphs as simple or complex, based on subgraphs' topological properties, the subgraphs are indexed. When a query is aligned, the seeds are extracted from the query and then located in the different subgraphs using the previously created indexes. At this point, the actual alignment is performed in parallel onto the different subgraphs, leveraging the seed-and-extend logic. Specifically, for the simple subgraphs the extension is performed by exploiting parallel sequence to graph alignment algorithms suitable for DAGs, while for complex subgraphs parallel algorithms for non-acyclic graphs based on the shortest-path problem are leveraged. Finally, the local results are reunified to obtain the final output, i.e., the best alignments of each query to the entire reference character-labeled graph.

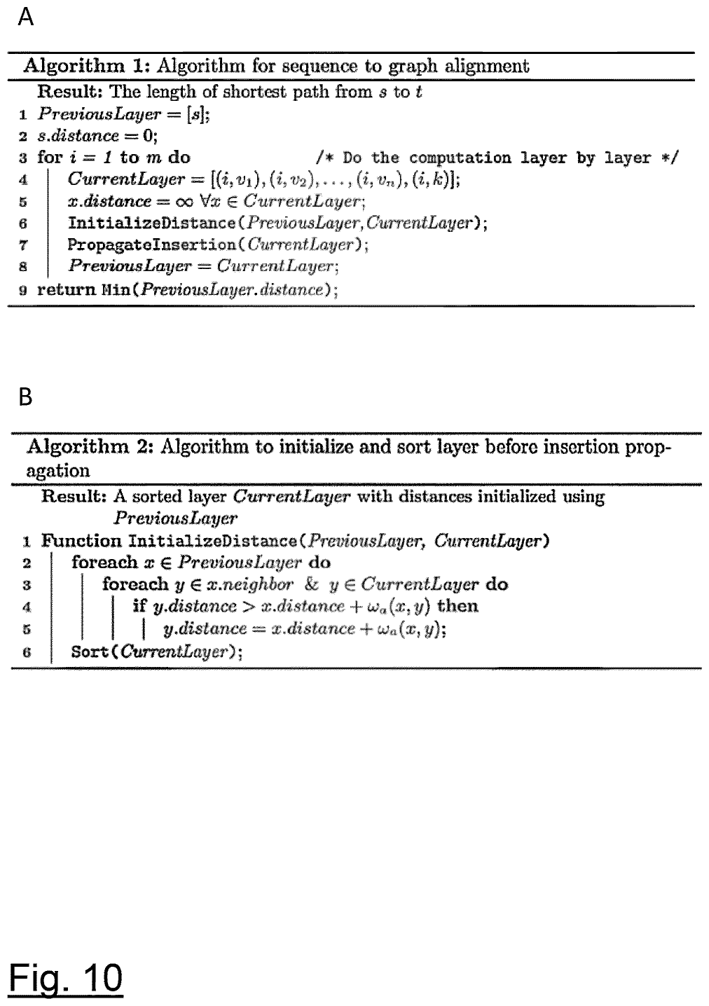

In an embodiment, according to the flow chart in FIG. 9 said computing step C is executed using a local adaptive approach where the character-labeled graph is segmented (i.e., partitioned) into simple regions and complex regions, i.e., a series of connected subgraphs, where the formers are Directed Acyclic Graphs, and the latter are Directed Non-Acyclic Graphs. After “labeling” the different subgraphs as simple or complex, based on subgraphs' topological properties, when a query is aligned, the alignment is performed in parallel onto the different subgraphs. Specifically, for the simple subgraphs the alignment is performed by exploiting parallel sequence to graph alignment algorithms suitable for DAGs (e.g., HGA's algorithm [7]), while for complex subgraphs the proposed system is employed, leveraging algorithms for non-acyclic graphs based on the shortest-path problem. Finally, the local results are reunified to obtain the final output, i.e., the best alignments of each query to the entire reference character-labeled graph.

In an embodiment, said computing step C is performed employing an alignment algorithm suitable for non-acyclic character-labelled reference genome graphs, which is the here proposed parallel version of the algorithm by Jain et al. [8].

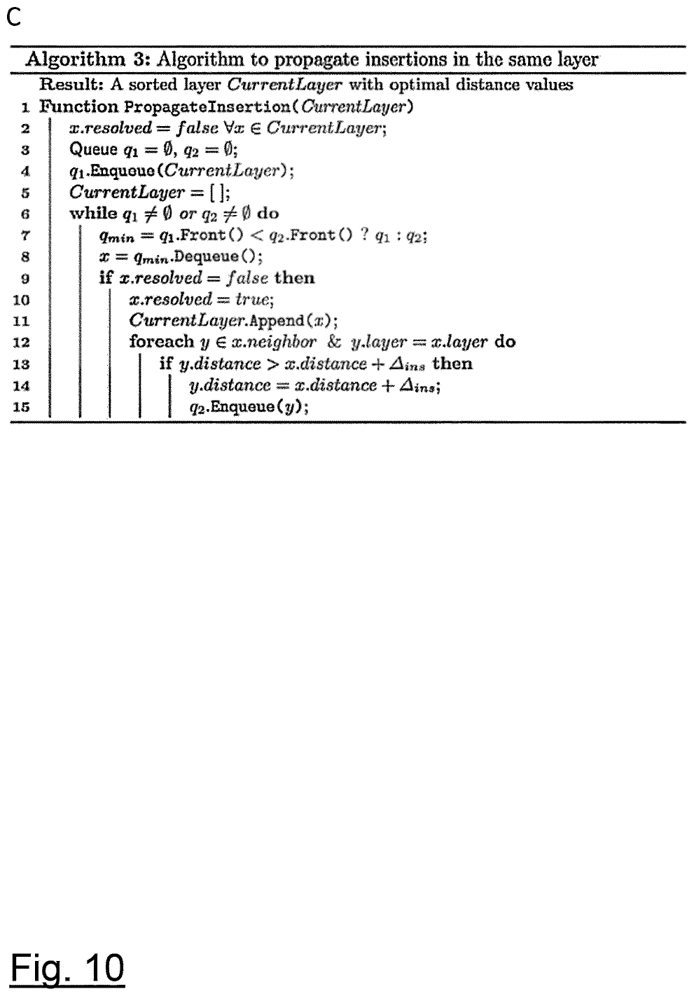

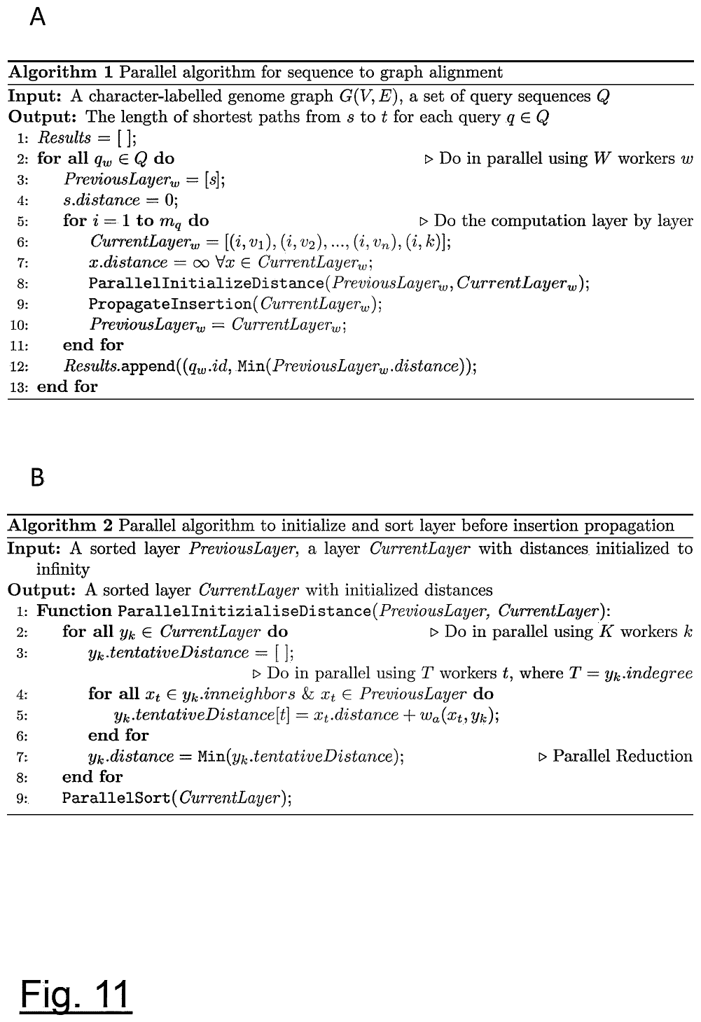

The algorithm according to the state of the art [8] computes the alignment of genomic sequences to reference genome graphs in O(|V|+m|E|) time, where m denotes the query size, and V and E denote the vertex and edge set of the graph, respectively. With reference to FIG. 10, Example 1 summarises, for comparative purpose, the main steps of the method proposed by Jain et al. [8]. The algorithm according to the state of the art is sequential, and it does not expose any form of intra-sequence parallelism. Vice versa, according to this embodiment, a ParallelInitializeDistance stage enables said intra-sequence parallelism, reverting the paradigm employed to navigate the alignment graph. Instead of propagating the previous layer distances to current layer nodes, through outgoing edges, here it is firstly proposed to retrieve current layer distances accessing previous layer's nodes, through ingoing edges, as reported in lines 2 and 4 of the Algorithm 2 in FIG. 11B. This modification, together with the dual representation of edges that provides fast access to both ingoing and outgoing edges, allows to parallelise the InitializeDistance (FIG. 10B, for comparative purpose) stage in an efficient way. The threads on a GPU can be executed in parallel, where each thread is responsible for the computation of a tentative distance for the considered node in the current layer, starting from the distance value of a certain in-neighbour from the previous layer. Then, the minimum distance, i.e., the best tentative distance for the considered node, is computed through parallel reduction on the tentative distance array, as shown in line 7 of the Algorithm 2 in FIG. 11B. By reverting the paradigm employed to navigate the alignment graph, the scores can be correctly propagated in parallel without incurring in race conditions and other inefficiencies. After all the final tentative distances have been computed, the current layer is sorted through a parallel sorting algorithm on the GPU, as shown in line 9 of the Algorithm 2 in FIG. 11B. Preferably, the parallel sorting algorithm is a poset sorting algorithm, best suited for partially ordered set of nodes. Also, the PropagateInsertion stage (Algorithm 3 in FIG. 10C) is executed on the GPU, to avoid excessive data transfer between CPU and GPUs that, otherwise, would require to transfer a whole layer of the alignment graph from the GPUs to the CPU, and vice versa, for each of the m iterations of the alignment algorithm.

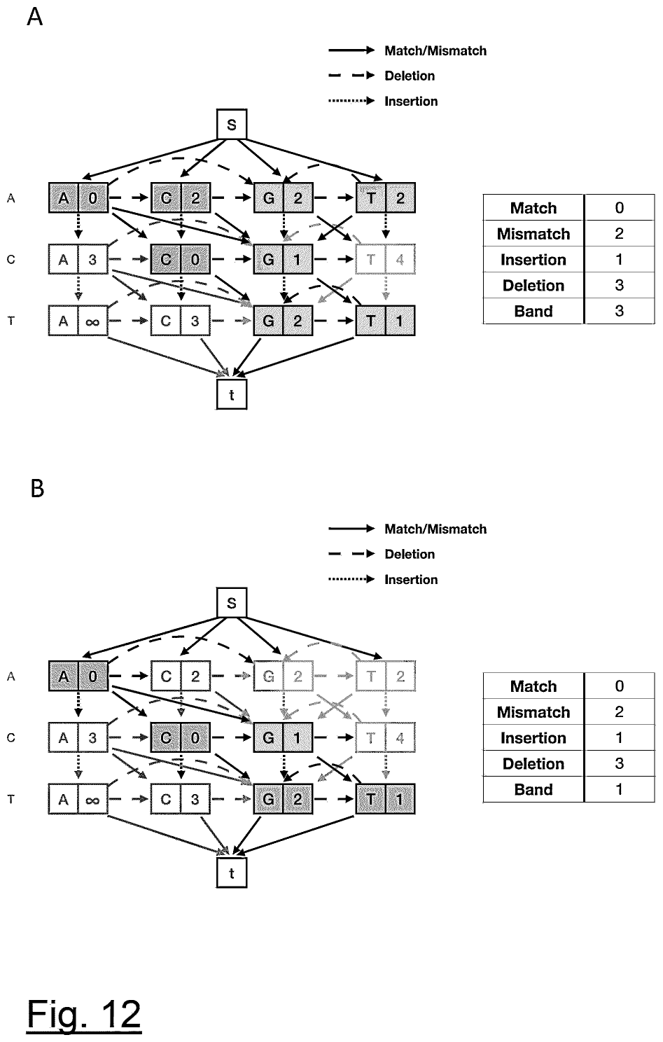

In a further embodiment, said computing step C is performed by leveraging an adaptive bandwidth heuristic that here is firstly proposed to speed up the execution of the said parallel alignment algorithm for each query sequence, by avoiding exploring those paths in the alignment graph that are clearly inconvenient w.r.t. the momentary best path, that is the path identifying the best alignment for the initial part of the query sequence. The adaptive bandwidth heuristic searches only for alignments that present a limited number of edits between the query sequence and the character-labeled graph. According to the present invention, this is accomplished at each iteration of the alignment algorithm by avoiding propagating the scores from the alignment graph's nodes whose alignment score differs more than a threshold value k from the best alignment score, i.e., the minimum distance in the alignment graph up to a certain iteration. As shown in FIG. 12, the adaptive bandwidth heuristics allows to reduce the number of explored paths in the alignment graph by avoiding computing the alignment scores for a subset of the alignment graph's nodes, while ensuring flexibility through the possibility of choosing the threshold value k.

The here proposed solution focuses on the preliminary steps preceding the execution of the actual alignment algorithm, where the state-of-the-art method never proposed a well-defined approach for such steps. The present invention allows to correctly handle the cases of alignment in presence of VNTRs and CNVs, and to predispose the parallelization of the alignment algorithm. Moreover, in an embodiment, the present invention enables to speed up the alignment process for non-acyclic genome graphs by leveraging the seed-and-extend paradigm and a local adaptive approach, which chooses the most efficient alignment algorithm on the base of local properties of the genome graphs. In addition, the here proposed novel formulation of the algorithm by Jain et al. [8] enables to efficiently compute the traceback of the best alignment, and to leverage the intra-sequence parallelism to accelerate the computation of each single alignment through the usage of a heterogeneous system equipped with GPU(s).

Finally, in an embodiment, the here proposed heuristic effectively allows to reduce the computational workload of the alignment algorithm to further improve its performance in terms of execution time.

Advantageously, the process according to the present invention is more accurate than alternative technologies suitable only for acyclic reference genome graphs, and more efficient than current solutions that support non-acyclic graphs but are not suitable for parallel execution on a heterogeneous system equipped with GPU(s).

### EXAMPLES

### Example 1: The State-of-the-Art Method (Comparative)

With reference to FIG. 10, are here summarised the main step of the method proposed by Jain et al. [8].

The main loop, shown in Algorithm 1, FIG. 10A, is made of two stages that are invoked m times until the optimal distances are known for the last layer, where m is the number of characters in the query sequence to be aligned. There is no need to store the alignment graph entirely, it is sufficient to store the information of two layers for each iteration, the Current Layer and the Previous Layer.

The input to the first stage, InitializeDistance, is the array of vertices, sorted in nondecreasing order of their distances in the previous layer. This stage (Algorithm 2, FIG. 10B) computes the tentative distances of all vertices in the current layer by using shortest distances computed for the previous layer, ignoring the insertion-weighted edges during the computation. It outputs the sorted tentative distances as an input to the second stage PropagateInsertion. The PropagateInsertion stage (Algorithm 3, FIG. 10C) computes the optimal distances of all vertices in the current layer while maintaining the sorted order for a subsequent iteration. To do that, it adjust the distances of the neighbours of vertex v such that they are no more than v.distance+Δᵢₙₛ. At the end of the m iterations, the shortest path from the source s to the sink t represents the optimal alignment of the query to the reference graph. Further details about the original algorithm are available in the work by Jain et al. [8].

### Example 2: Alignment with Intra- and Inter-Sequence Parallelism (According to the Invention)

With reference to FIG. 6 and FIG. 11, in this embodiment, the method is based on a modified parallel version of the described Jain et al. algorithm, that can be executed on heterogeneous computing systems, containing at least a CPU (Central Processing Unit) and a GPU (Graphics Processing Units). This is enabled by the dual representation of the edges in the char-labelled sequence graph.

In this embodiment, the method utilises both the CPU(s) and GPU(s) for sequence-to-graph alignment, employing both inter-sequence and intra-sequence parallelism.

To realise inter-sequence parallelism, a CPU thread is assigned to a GPU stream, and each couple is responsible for the alignment of 1 query sequence at a time. Depending on the size of the graph, and on the resource availability of the GPUs in the system, each GPU can execute one or more streams at the same time (at least one stream per GPU). An additional main CPU thread is responsible for load balancing, which is done by dynamically scheduling alignments to a thread/stream couple, for example according to the work stealing strategy, where idle threads fetch from the remaining queries to be aligned. Each CPU thread is responsible for data transfer to/from the corresponding GPU stream. This enables asynchronous memory transfer (computation and data transfer overlap in time) and avoids the need of communication between GPU streams, since the alignments of different queries are independent.

Jain et al. [8] algorithm is not suitable for intra-sequence parallelism on GPU. To enable that, the ParallelInitializeDistance stage according to the present invention reverts the paradigm employed to navigate the alignment graph. Instead of propagating the previous layer distances to current layer nodes, through outgoing edges, here it is firstly proposed to retrieve current layer distances accessing previous layer's nodes, through ingoing edges, as reported in lines 2 and 4 of the Algorithm 2 in FIG. 11B. This modification, together with the dual representation of edges, allows to parallelise the InitializeDistance stage (Algorithm 2 in FIG. 10B) in an efficient way. Each thread on the GPU is responsible for the computation of a tentative distance for the considered node in the current layer, starting from the distance value of a certain in-neighbour from the previous layer. The tentative distances for each node are saved in an array of size equal to the max in-degree in the graph. Then, the minimum distance, i.e., the final tentative distance for the considered node, is computed through parallel reduction on the tentative distance array, as shown in line 7 of the Algorithm 2 in FIG. 11B. After all the final tentative distances have been computed, the current layer is sorted through a parallel sorting algorithm on the GPU (line 9 of the Algorithm 2 in FIG. 11B). Preferably, the parallel sorting algorithm is a poset sorting algorithm, best suited for partially ordered set of nodes. Also, the PropagateInsertion stage (Algorithm 3 in FIG. 10C) is executed on the GPU, to avoid excessive data transfer between CPU and GPUs that, otherwise, would require to transfer a whole layer of the alignment graph from the GPUs to the CPU, and vice versa, for each of the m iterations of the alignment algorithm, where m is the length of the query sequence to be aligned.

### Example 3: Computing the Best Alignments Between a Set of Query Sequences and a Non-Acyclic Genome Graph, and the Traceback Associated to them (According to the Invention)

With reference to FIG. 7, in this embodiment, for each char-labelled node of the alignment graph, the method stores, in addition to the edit distance between the query and the shortest path in the alignment graph reaching that node, two fields:
the Path;
a CIGAR string.

These fields are updated every time the distance of a certain node is updated, for example in the ParallelInitializeDistance stage (line 7 of the Algorithm 2 in FIG. 11B), or in the PropagateInsertion stage (line 14 in Algorithm 3, FIG. 10C).

The Path of a node is updated by appending the considered node ID to the Path of its predecessor in the shortest path. For instance, considering node 3, with Path=[1, 2, 3], supposing that node 3 propagates its distance to node 5 during the execution of the alignment, either in ParallelInitializeDistance or in PropagateDistance stages, node 5 would have Path=[1, 2, 3, 5].

The CIGAR string of a node is updated similarly, by adding to the CIGAR of its predecessor in the shortest path the information about the kind of edge connecting the predecessor to the considered node in the alignment graph. For instance, considering node X, with CIGAR=‘4M2D3M’, representing an alignment with 4 (mis)matches, 2 deletions and other 3 (mis)matches, supposing that node X propagates its distance to node Y during the execution of the alignment, either in ParallelInitializeDistance or in PropagateDistance stages, node Y would have:
CIGAR=‘4M2D4M’, if X is connected to Y through a (mis)match-weighted edge in the alignment graph;
CIGAR=‘4M2D3M1D’, if X is connected to Y through a deletion-weighted edge in the alignment graph;
CIGAR=‘4M2D3M1I’, if X is connected to Y through an insertion-weighted edge in the alignment graph.

At the end of the execution, the nodes with the shortest distance (best alignment score) will also contain the Path and CIGAR fields, constituting the traceback of the corresponding best alignment.

### Example 4: Computing Sequence to Graph Alignment Leveraging an Adaptive Bandwidth Heuristic (According to the Invention)

With reference to FIG. 12, in this embodiment, the here proposed heuristic is employed to speed up the execution of the alignment algorithm by avoiding exploring those paths in the alignment graph that are clearly inconvenient w.r.t. the momentary best path, that is the path identifying the best alignment for the initial part of the query sequence. The adaptive bandwidth heuristic searches only for alignments that present a limited number of edits between the query sequence and the character-labeled graph, by avoiding propagating the scores from the alignment graph's nodes whose alignment score differs more than a threshold value k from the best alignment score, i.e., the minimum distance in the alignment graph up to a certain iteration. FIG. 12 shows examples of how the adaptive bandwidth heuristics allows to reduce the number of explored paths in the alignment graph by avoiding computing the alignment scores for a subset of the alignment graph's nodes, while ensuring flexibility through the possibility of choosing the band value k. FIG. 12A shows the scores propagated in the case of a band value equal to 3, while FIG. 12B shows the scores propagated in the case of a band value equal to 1. Only the scores in the cells colored in gray are propagated towards the edges colored in black, while the scores in the white cells are not propagated, resulting in a reduced execution time. It is possible to notice how a lower band value k results in a lower number of propagated scores, thus in a shorter execution time. Obviously, using a band value that is too low could result in suboptimal alignments for some query sequences. However, this is not the case of the examples in FIGS. 12A and 12B, where the heuristic alignment algorithm is still able to find the globally optimal alignment, as in the case of the exact algorithm.

### Example 5: Runtime Performance Characterization

The evaluation experiments according to the process as defined in Example 2 were executed on a 2.6 GHz Intel Core i7-8850H with 16 GB of DDR4 RAM at 2400 MHz, and a NVIDIA Geforce GTX 1660 with 6 GB of GDDR5 RAM at 2000 MHz.

The proposed implementation takes as input a set of sequenced reads in FASTA/Q format (both compressed with gzip or uncompressed), and a reference genome graph in Graphical Fragment Assembly (GFA) format. For our internal character-labelled graph structure, we chose a dual representation employing the popular Compressed Sparse Row (CSR) and Compressed Sparse Column (CSC) format.

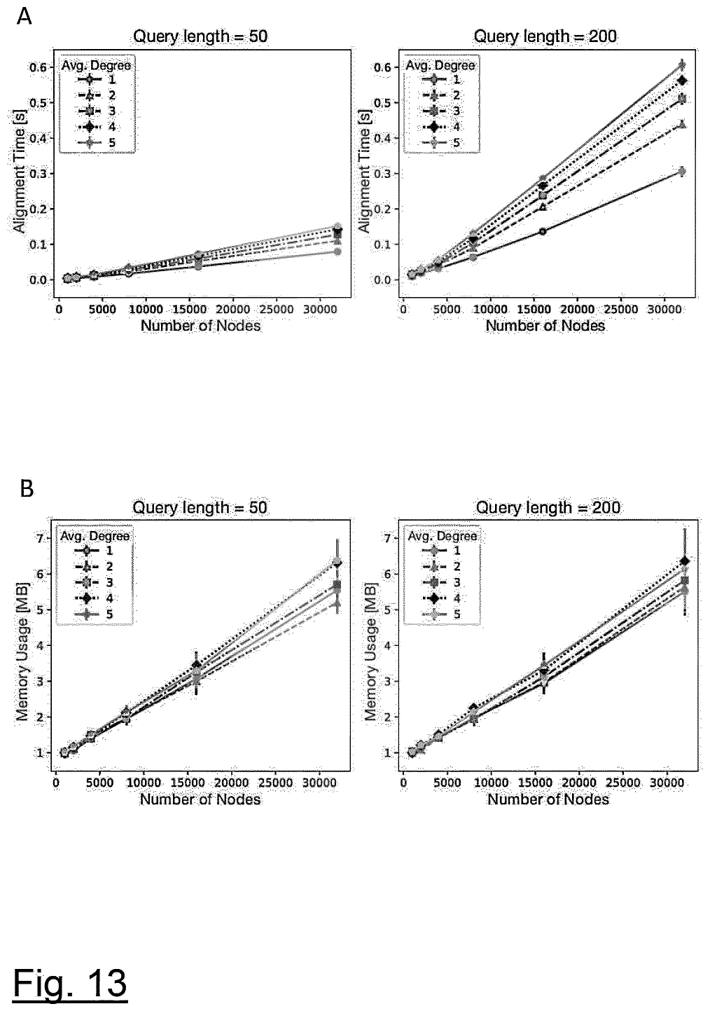

To characterise our implementation's performance in terms of execution time and memory footprint, we created a set of 30 synthetic graphs, uniformly distributed over the alphabet={A, C, G, T}. We tested different combinations of number of nodes (1000, 2000, 4000, 8000, 16000, 32000), average degree (1, 2, 3, 4, 5), and query length (50 bp, 100 bp, 150 bp, 200 bp). For each possible combination of these parameters, we conducted 10 different alignment runs to obtain robust results. FIG. 13A presents the scaling of the alignment time w.r.t. the number of nodes of the genome graph and its average degree. The execution time of our implementation scales almost linearly with the query length and the number of nodes and edges, in accordance with the theoretical complexity O(|V|+m|E|). In addition, FIG. 13B illustrates how the memory usage scales with the number of nodes and the average degree of the genome graph, showing that RAM consumption scales linearly with the size of the vertices set. Moreover, we can see how the memory footprint of our implementation is unaffected by the query's length and the degree of the genome graph, in accordance with the theoretical space complexity O(|V|).

### Example 6: Evaluation on Sars-CoV2 Genome Data

To test the effectiveness of our work on a real-life workload, we also compared the runtime performances of our implementation to the two optimal aligners that currently support non-acyclic graphs: GraphAligner and AStarix.

We employed VG-toolkit to build a variation graph from the SARS-CoV-2 reference sequence, and the 2019-nCoV_total set of mutations by CNCB. In this way, we created a highly branched non-acyclic graph of real viral variations, with 39253 nodes 95440 edges. We then selected 5 random sets of 10000 real SARS-CoV-2 reads with an average length ˜100 bp. The same alignment experiment, consisting in computing the alignment score between each sequence read and the previously built variation graph, was repeated 10 times for each tested aligner, to produce robust results. Table I reports the outcome of such evaluation, showing that our implementation obtains an average 2.17× speed-up over GraphAligner's optimal algorithm, while reducing memory consumption by 61.9× on average. Instead, AStarix was never able to complete the alignment task, as it gives SegFault error during the construction of its internal trie, probably due to SARS-CoV-2 variation graph's complexity.

TABLE 1

| | \|V\| | \|E\| | Align. Time [s] | RAM [MB] |
|---|---|---|---|---|
| This work | 51780 | 107967 | 4012.4 ± 31.2 | 16.4 ± 0.15 |
| GraphAligner | 78506 | 190880 | 8724.7 ± 98.3 | 1010.13 ± 8.21 |
| AStarix | N.A. | N.A. | N.A. | N.A. |

The results in term of where the mutations have been identified on the graph and the corresponding scores, obtained with the process according to the present invention overlap with the results obtained with the state-of-the-art algorithms. This was expected, since they are both exact algorithms, but it is surprising the better performance as above detailed observed when applying the method according to the present invention.

### Example 7: Evaluation on a System with Multiple GPUs

The evaluation experiments according to the process as defined in Example 2 were executed on a 2.0 GHZ Intel Xeon Platinum with 768 GB of DDR4 RAM at 2400 MHz, and 8 NVIDIA V100, each equipped with 16 GB of HBM2 RAM at 876 MHz.

The proposed implementation takes as input a set of sequenced reads in FASTA/Q format (both compressed with gzip or uncompressed), and a reference genome graph in Graphical Fragment Assembly (GFA) format. For our internal character-labelled graph structure, we chose a dual representation employing the popular Compressed Sparse Row (CSR) and Compressed Sparse Column (CSC) format.

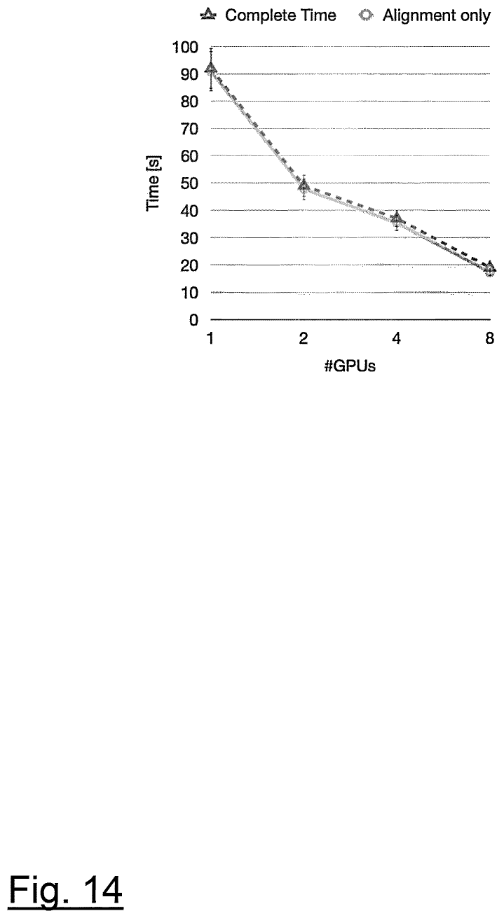

To characterise our implementation's performance in terms of execution time scaling w.r.t. the number of employed GPUs, we used the SARS-CoV-2 variation graph from Example 6, and 5 random sets of 10000 real SARS-CoV-2 reads with an average length ˜100 bp. We tested different configurations, employing 1, 2, 4 or 8 GPUs available in the system. The same alignment experiment, consisting in computing the alignment score and its traceback between each sequence read and the SARS-CoV-2 variation graph, was repeated 10 times for each configuration, to produce robust results. FIG. 14 presents the scaling of the alignment time and of the complete execution time, comprising also the creation of the needed data structures, w.r.t. the number of employed GPUs. We can see how both the alignment time and the complete execution time scales well with the number of GPUs. Also, it is possible to see how the overhead related to the creation of the needed data structures slightly grows with the number of GPUs, since multiple copies of such data structures are needed. However, the overhead is still negligible w.r.t. to the alignment time, confirming the benefit of employing multiple GPUs.

### Example 8: Evaluation of the Proposed Heuristic

The evaluation experiments according to the process as defined in Example 2 and Example 4 were executed on a 2.0 GHZ Intel Xeon Platinum with 768 GB of DDR4 RAM at 2400 MHz, and 8 NVIDIA V100, each equipped with 16 GB of HBM2 RAM at 876 MHZ.

The proposed implementation takes as input a set of sequenced reads in FASTA/Q format (both compressed with gzip or uncompressed), and a reference genome graph in Graphical Fragment Assembly (GFA) format. For our internal character-labelled graph structure, we chose a dual representation employing the popular Compressed Sparse Row (CSR) and Compressed Sparse Column (CSC) format.

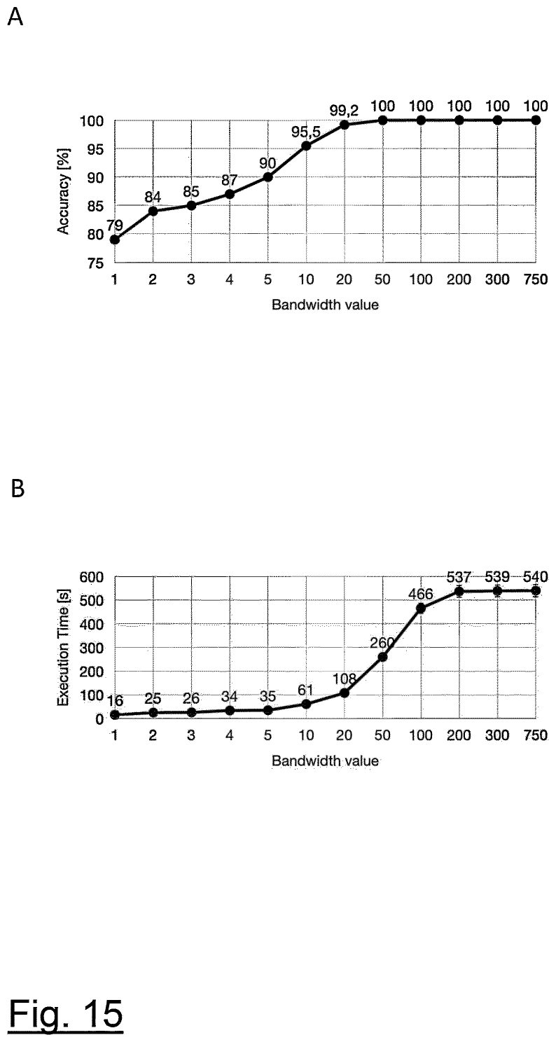

To characterise the trade-off between accuracy and execution time provided by the proposed heuristic, we used the SARS-CoV-2 variation graph from Example 6, and a set of 1000 real SARS-CoV-2 reads with an average length of ˜500 bp, where 792 reads perfectly matched at least a path in the variation graph. We tested different values of the threshold k employed in our heuristic, and we measured the execution time and the percentage of best alignments computed correctly. In detail, we tested the following values for the threshold: 1, 2, 3, 4, 5, 10, 20, 50, 100, 200, 300, 750. FIG. 15A and FIG. 15B respectively show the achieved level of accuracy and the execution time for the heuristic alignment algorithm executed with different bandwidth values. We can notice how a k value of 1, which means that every path with a distance higher than the best plus one is discarded, our implementation reaches a level of accuracy of 79.2%. This result is correct because it means that it found all the perfectly matching reads (792 over 1000), with a speedup of ˜33× w.r.t. the exact implementation. Other important results are obtained with the bandwidth value of 10 (˜9× speedup and 95.5% of accuracy), 20 (˜5× speedup and 99.2% of accuracy), and 50 (˜2× speedup and 100% of accuracy). The efficiency of the heuristics is then confirmed because with this last value the process is significantly faster while guaranteeing the perfect mapping of all the reads.

### BIBLIOGRAPHIC REFERENCES

[1] Rakocevic, Goran, et al. “Fast and accurate genomic analyses using genome graphs.” Nature genetics 51.2 (2019): 354-362
[2] Garrison, Erik, et al. “Variation graph toolkit improves read mapping by representing genetic variation in the reference.” Nature biotechnology 36.9 (2018): 875-879.
[3] Rautiainen, Mikko, and Tobias Marschall. “GraphAligner: rapid and versatile sequence-to-graph alignment.” Genome biology 21.1 (2020): 1-28.
[4] C. Jain, S. Misra, H. Zhang, A. Dilthey and S. Aluru, “Accelerating Sequence Alignment to Graphs,” 2019 IEEE International Parallel and Distributed Processing Symposium (IPDPS), 2019, pp. 451-461, doi: 10.1109/IPDPS.2019.00055.
[5] Rätsch, Gunnar, and Martin Vechev. “AStarix: Fast and Optimal Sequence-to-Graph Alignment.” Research in Computational Molecular Biology: 24th Annual International Conference, RECOMB 2020, Padua, Italy, May 10-13, 2020, Proceedings. Vol. 12074. Springer Nature, 2020.
[6] Ivanov, Pesho, Benjamin Bichsel, and Martin Vechev. “Fast and Optimal Sequence-to-Graph Alignment Guided by Seeds.” bioRxiv (2021).
[7] Feng, Zonghao, and Qiong Luo. “Accelerating sequence-to-graph alignment on heterogeneous processors.” 50th International Conference on Parallel Processing. 2021.
[8] C. Jain, H. Zhang, Y. Gao, and S. Aluru, “On the complexity of sequence-to-graph alignment,” Journal of Computational Biology, vol. 27, no. 4, pp. 640-654, 2020.
[9] Rautiainen, Mikko, Veli Makinen, and Tobias Marschall. “Bit-parallel sequence-to-graph alignment.” Bioinformatics 35.19 (2019): 3599-3607.

1. A system for computing the best alignment score between a set of query sequences and a non-acyclic sequence-labelled reference genome graph comprising a processor and non-transitory memory, wherein the memory comprises instructions that, when executed, cause the processor to:
A. Transforming the non-acyclic sequence-labelled reference genome graph, which comprises nodes and different paths, in a character-labelled graph by
A1: duplicating string-labelled nodes, if any, that appear in different paths, both in their original and reverse complement strands;
A2: unrolling each string-labelled node to form a linear subgraph of contiguous character-labelled nodes;
A3: analysing the node sequences to search for repeats of subsequences, if any, that can be represented as cycles in the character-labelled sub-graph, to guarantee the finest granularity when handling genetic variations involving repetitions, e.g., tandem repeats;
A4: Connecting said character-labelled sub-graphs each other using the information contained in the edges of the non-acyclic sequence-labelled genome graph, i.e., source node, target node, overlap, wherein an edge is created between the last character-labelled node extracted from the source node, and the character-labelled node corresponding to the character in the i-th position of the target node's sequence, where i is equal to the length of the overlap+1, then another edge is created between the last character-labelled node preceding the overlap in the source node, and the first character-labelled node of the target node's sequence, obtaining a character-labelled reference graph;
B. Creating a concurrent dual representation of the character-labelled reference graph, made by a single copy of nodes' information, and two copies of edges' information employing two complementary representations that provide concurrent fast access to the in-neighbours and out-neighbours of a certain node, respectively;
C. Computing the alignment score between each query sequence of length m and the dually represented character labelled reference graph, wherein said computing step comprises the creation of the alignment graph, composed by a source vertex s, a sink vertex t, and multiple layers given by m repetitions of a character-labelled graph, called sequence graph, and the alignment is performed by searching for the shortest path from the source vertex s to the sink vertex t, leveraging both inter-sequence and intra-sequence parallelism.

2. The system according to claim 1, wherein said representations according to step B are selected in the group consisting of:
two adjacency lists storing, for each node, the list of in-neighbours and the list of out-neighbours;
an adjacency matrix saved both in a row-major (out-neighbours) and column-major (in-neighbours);
a compressed adjacency matrix, stored using both the Compressed Sparse Row (CSR) representation (out-neighbours) and the Compressed Sparse Column (CSC) representation (in-neighbours).

3. The system according to claim 1, wherein said computing is performed employing an alignment algorithm suitable for non-acyclic graph executed on a heterogeneous computing system, containing at least a CPU (Central Processing Unit) and a GPU (Graphics Processing Units).

4. The system according to claim 3, wherein both the CPU(s) and GPU(s) are used for executing the sequence-to-graph alignment algorithm, employing both inter-sequence parallelism and intra-sequence parallelism.

5. The system according to claim 1, wherein said computing step is employed for computing the best alignments between a set of query sequences and a non-acyclic genome graph, and also the traceback associated to them.

6. The system according to claim 5, wherein for each character-labelled node of the alignment graph, the method requires to store, in addition to the edit distance between the query and the shortest path in the alignment graph reaching that node, two fields:
an array named Path, containing the ordered sequence of IDs of the nodes visited in the shortest path culminating to the considered nodes;
a CIGAR string, describing the complete sequence of edits the sub-query, i.e., subsequence of the query sequence up to position i, where i is the current iteration of the alignment algorithm, should undergo to match the sequence extracted by navigating the graph through the identified Path, wherein, at the end of the execution, the nodes with the shortest distance comprise the Path and CIGAR fields, constituting the traceback of the corresponding best alignment.

7. The system according to claim 6, wherein said Path and said CIGAR string are updated every time the distance of a certain node is updated during the execution of the alignment algorithm based on shortest-path search.

8. The system according to claim 7, wherein the Path of a node is updated by appending the considered node ID to the Path of its predecessor in the shortest path.

9. The system according to claim 7, wherein the CIGAR string of a node is updated by adding to the CIGAR of its predecessor in the shortest path the information about the kind of edge connecting the predecessor to the considered node in the alignment graph.

10. The system according to claim 1, wherein said step C is executed through a seed-and-extend approach comprising:
a. the character-labeled graph is indexed by leveraging graph indexes suitable for non-acyclic graphs;
b. subsequences, called seeds, are extracted from the query sequence using a moving window of fixed or variable length onto the query sequence;
c. candidate alignment locations are retrieved by mapping the previously extracted seeds onto the graph by leveraging previously built graph indexes;
d. the complete alignment of the query sequence is computed by extending the match of all the seeds, or a subset of them, using a parallel alignment algorithm based on shortest-path search in the alignment graph, where the first layer of the alignment graph contains only the nodes corresponding to the last character of each mapped seed.

11. The system according to claim 10, wherein, said step C is executed through a local adaptive approach wherein the character-labeled graph is:
segmented (i.e., partitioned) into simple regions and complex regions, i.e., a series of connected subgraphs, where the formers are Directed Acyclic Graphs, and the latter are Directed Non-Acyclic Graphs;
said subgraphs are labelled as simple or complex;
said labelled subgraphs are indexed;
the seeds are extracted from the query and then located in the different subgraphs using said indexes;
the alignment is performed in parallel onto the different subgraphs, leveraging the seed-and-extend logic, wherein for the simple subgraphs said extension is performed by exploiting parallel sequence to graph alignment algorithms suitable for DAGs and wherein for complex subgraphs said extension is performed leveraging parallel algorithms for non-acyclic graphs based on the shortest-path problem;
the local results are reunified to obtain the final output, i.e., the best alignments of each query to the entire reference character-labeled graph.

12. The system according to claim 1, where said step C is executed through a local adaptive approach comprising:
a. the character-labeled graph is segmented (i.e., partitioned) into simple regions and complex regions, i.e., a series of connected subgraphs, where the formers are Directed Acyclic Graphs, and the latter are Directed Non-Acyclic Graphs;
b. the different subgraphs are labelled as simple or complex;
c. the alignment is performed in parallel onto the different subgraphs wherein, for the simple subgraphs, the alignment is performed by exploiting parallel sequence to graph alignment algorithms suitable for DAGs, and wherein for complex subgraphs the alignment is performed leveraging parallel algorithms for non-acyclic graphs based on the shortest-path problem;
d. the local results are reunified to obtain the final output, i.e., the best alignments of each query to the entire reference character-labeled graph.

13. The system according to claim 1, wherein the parallel alignment algorithm based on the shortest-path search problem is a parallel formulation that leverages both inter-sequence and intra-sequence parallelism, wherein current layer distances are retrieved by accessing previous layer's nodes through ingoing edges, and tentative distances for the considered node in the current layer are computed in parallel, starting from the distance value of in-neighbours from the previous layer, then, the minimum distance is computed through parallel reduction on the tentative distances.

14. The system according to claim 1, wherein an adaptive bandwidth heuristic is employed, by not propagating the scores from the previous layer to the current layer of the alignment graph for those nodes whose alignment score differs more than a threshold value k from the best alignment score, that is the minimum distance in the alignment graph up to a certain iteration, searching only for alignments presenting a limited number of edits between the query sequence and the character-labeled graph.
